SEQUENCE LISTING (12 sequences)

SEQ ID NO: 1 - DNA, 20 bp, Artificial Sequence
  synthetic oligonucleotide
tccatgacgttcctgacgtt

SEQ ID NO: 2 - DNA, 8 bp, Artificial Sequence
  synthetic oligonucleotide
tcaacgtt

SEQ ID NO: 3 - DNA, 6 bp, Artificial Sequence
  synthetic oligonucleotide
gacgtt

SEQ ID NO: 4 - DNA, 6 bp, Artificial Sequence
  synthetic oligonucleotide
agcgtt

SEQ ID NO: 5 - DNA, 6 bp, Artificial Sequence
  synthetic oligonucleotide
aacgct

SEQ ID NO: 6 - DNA, 6 bp, Artificial Sequence
  synthetic oligonucleotide
aacgat

SEQ ID NO: 7 - DNA, 9 bp, Artificial Sequence
  synthetic oligonucleotide
ttcaacgtt

SEQ ID NO: 8 - DNA, 7 bp, Artificial Sequence
  synthetic oligonucleotide
tgacgtt

SEQ ID NO: 9 - DNA, 7 bp, Artificial Sequence
  synthetic oligonucleotide
tagcgtt

SEQ ID NO: 10 - DNA, 7 bp, Artificial Sequence
  synthetic oligonucleotide
taacgct

SEQ ID NO: 11 - DNA, 7 bp, Artificial Sequence
  synthetic oligonucleotide
taacgat

SEQ ID NO: 12 - DNA, 20 bp, Artificial Sequence
  synthetic control oligonucleotide
tccaggacttctctcaggtt

(19) United States
(12) Patent Application Publication
(10) Pub. No.: US 2003/0119774 A1
(43) Pub. Date: Jun. 26, 2003

(54) Compositions and methods for stimulating an immune response

(76) Inventors: Marianna Foldvari, Saskatoon (CA); Maria Baca-Estrada, Ottawa (CA)

Correspondence Address: PERKINS COIE LLP, P.O. BOX 2168, MENLO PARK, CA, 94026, US

(21) Appl. No.: 10/254,102

(22) Filed: Sep. 23, 2002

Related U.S. Application Data
(60) Provisional application No. 60/325,124, filed on Sep. 25, 2001.

(51) Int. Cl. A61K 48/00; A61K 39/00

(52) U.S. Cl. 514/44; 424/185.1

(57) ABSTRACT

A composition for improving the immune response in a subject is described. The composition includes biphasic lipid vesicles associated with an immunogen. In one embodiment a nucleic acid containing at least one cytosine-guanine (CpG) dinucleotide is associated with the lipid vesicles to achieve a synergistic immune response.

This application claims the benefit of U.S. Provisional Application No. 60/325,124 filed Sep. 25, 2001, which is incorporated herein by reference in its entirety.

### FIELD OF THE INVENTION

The present invention relates to compositions, kits, and methods for eliciting an immune response. More particularly, the invention relates to a lipid vesicle composition and to a lipid vesicle composition in combination with an oligonucleotide having a cytosine-guanine (CpG) dinucleotide motif, for eliciting an immune response to an antigen.

### REFERENCES

Chanock, R. M., Lerner, R. A., Brown, F. and Ginsberg, H, “New Approaches to Immunization, Vaccines” 86, Cold Spring Harbor, N.Y. (1987).

Klinman, D. M. et al., Vaccine, 17:19 (1999).

Kreig, A. M. et al., Pharmacology & Therapeutics, 84:113 (1999).

### BACKGROUND OF THE INVENTION

Vaccines have traditionally consisted of live attenuated pathogens, whole inactivated organisms, or inactivated toxins. Although these have proved successful in the past, several drawbacks have limited their use against more challenging diseases such as hepatitis C or AIDS. First, certain live-attenuated vaccines can cause disease in immunosuppressed individuals by reverting to a more virulent phenotype. Second, whole inactivated vaccines (e.g., Bordetella pertussis) contain reactogenic components that can cause undesirable side effects. Third, some pathogens are difficult or even impossible to grow in culture (e.g., hepatitis B, hepatitis C, and human papillomavirus), making preparation of a vaccine problematic.

In the past decade, several new approaches to vaccine development have emerged that may have significant advantages over traditional approaches. These new approaches include recombinant protein subunits, synthetic peptides, and plasmid DNA. Although they offer advantages such as reduced toxicity, they are poorly immunogenic when administered alone. This is particularly true for vaccines based on recombinant proteins or peptides. Traditional vaccines are heterogeneous and contain many epitopes, some of which can provide additional T-cell help or function as adjuvants (e.g., bacterial DNA in whole-cell vaccines). Therefore, a great need exists for immunological adjuvants that are potent, safe, and compatible with new-generation vaccines, including DNA vaccines.

### SUMMARY OF THE INVENTION

Accordingly, it is an object of the invention to provide an adjuvant that achieves an enhanced immune response relative to the response achieved in the absence of the adjuvant.

It is another object of the invention to provide a lipid vesicle adjuvant.

It is a further object of the invention to provide an adjuvant comprised of a mixture of biphasic lipid vesicles and an oligonucleotide having a CpG motif.

It is yet another object of the invention to provide a method of improving the immune response achieved by administering an immunogen in combination with a biphasic lipid vesicle by further including a CpG oligonucleotide.

In one aspect, the invention includes a composition for eliciting in a subject an immune response to an immunogen. The composition includes a suspension of biphasic lipid vesicles having a central core compartment containing an oil-in-water emulsion, and, entrapped in the biphasic lipid vesicles, an immunogen.

In one embodiment, the immunogen is an antigen derived from bacterial, viral, parasitic, plant, or fungal origin.

The immunogen is effective to elicit a humoral immune response, or alternatively, is effective to elicit a cell-mediated immune response.

In another embodiment, the immunogen is admixed with the vesicles. In another embodiment, the immunogen is entrapped in the vesicles.

In a preferred embodiment, the composition further comprises an oligonucleotide comprising one or more cytosine-guanine (CpG) dinucleotides. Generally, the CpG oligonucleotide is of the form X₁ CG X₂, where X₁ and X₂ are nucleotides. More generally, the CpG oligonucleotide is of the form NₙX₁ CG X₂Nₘ, where X₁, X₂, Nₙ, and Nₘ are nucleotides, and n and m individually range from 0 to about 100. Exemplary CpG oligonucleotides include TCCATGACGTTCCTGACGTT (SEQ ID NO:1), TCAACGTT (SEQ ID NO:2), GACGTT (SEQ ID NO:3), AGCGTT (SEQ ID NO:4), AACGCT (SEQ ID NO:5), or AACGAT (SEQ ID NO:6), wherein C and G are unmethylated. In another embodiment, the CpG oligonucleotide sequences comprises a T nucleotide on its 5′ end, and exemplary sequences include TTCAACGTT (SEQ ID NO:7), TGACGTT (SEQ ID NO:8), TAGCGTT (SEQ ID NO:9), TAACGCT (SEQ ID NO:10), and TAACGAT (SEQ ID NO:11).

The CpG oligonucleotide sequence comprises typically between about 2 to about 250 nucleotides, more preferably 2-100 nucleotides, and still more preferably 8-100 nucleotides.

In yet another embodiment, the oligonucleotide has a phosphate backbone modification, such as a phosphorothioate backbone modification.

In another aspect, the invention includes a composition for eliciting in a subject an immune response to an immunogen. The composition comprises a suspension of biphasic lipid vesicles having a central core compartment containing an oil-in-water emulsion, and associated with the vesicles, (i) an immunogen and (ii) a CpG oligonucleotide.

The immunogen and the CpG oligonucleotide are admixed with the vesicles, in one embodiment. In another embodiment, the immunogen is entrapped in the vesicles. In still another embodiment, the CpG oligonucleotide is entrapped in the vesicles. In yet another embodiment, the immunogen and the CpG oligonucleotide are entrapped in the vesicles.

In another aspect, the invention includes a kit for preparation of a composition effective to elicit in a subject an immune response to an immunogen. The kit is comprised of (i) a biphasic lipid vesicle component; (ii) an immunogen component; and (iii) a CpG oligonucleotide component.

In another aspect, the invention includes a kit for preparation of a composition effective to elicit in a subject an immune response to an immunogen. The kit is comprised of (i) a first component of an immunogen entrapped in biphasic lipid vesicles and (ii) a second component of a CpG oligonucleotide. The two components are admixed to form a composition effective to elicit an immune response.

In another aspect, the invention includes a kit for preparation of a composition effective to elicit in a subject an immune response to an immunogen. The kit is comprised of (i) a biphasic lipid vesicle-entrapped CpG oligonucleotide; and (ii) an immunogen component. The two components are admixed to form a composition effective to elicit an immune response.

In another aspect, the invention includes an improvement in a composition comprised of a biphasic lipid vesicle and an immunogen. The improvement comprises including a CpG oligonucleotide in the composition. The improvement is effective to enhance the immune response to the immunogen relative to the response obtained by administration of the vesicles and the immunogen in the absence of the oligonucleotide.

In still another aspect, the invention includes a method for enhancing the immune response obtained by administration of a biphasic lipid vesicle entrapped immunogen, comprising administering a CpG oligonucleotide.

In one embodiment of this aspect, the lipid vesicles and the oligonucleotide are administered subcutaneously or mucosally.

These and other objects and features of the invention will be more fully appreciated when the following detailed description of the invention is read in conjunction with the accompanying drawings.

### BRIEF DESCRIPTION OF THE DRAWINGS

FIG. 1A is a bar graph showing the anti-OmIA IgG serum titre in pigs after subcutaneous administration of an antigen isolated from the outer membrane of Actinobacillus pleuropneumoniae (OmIA) in association with biphasic lipid vesicles (Group 1-2); or biphasic lipid vesicles plus CpG (Group 1-3). Group 1-1 and Group 1-4 are control groups.

FIG. 1B shows the lung pathology score following evaluation of a lung section from each test animal in the test groups of FIG. 1A, where Group 1-1 is represented by the closed squares, Group 1-2 by the closed triangles, Group 1-3 by the inverted closed triangles, and Group 1-4 by the closed diamonds.

FIG. 2 is a bar graph showing serum anti-OmIA IgG titer following subcutaneous immunization with saline (Group 2-1), the antigen OmIA in saline and a CpG oligonucleotide (Group 2-2); the antigen OmIA associated with biphasic lipid vesicles and a CpG oligonucleotide (Group 2-3); or OmIA in a mineral-based adjuvant (Group 2-4).

FIG. 3 is a bar graph showing the anti-gD IgG serum titre for mice immunized subcutaneously (SQ) or intranasally (IN) with viral antigen glycoprotein D (“gD antigen”; Group SQ-1 and Group IN-4); with gD antigen plus biphasic lipid vesicles (formulation no. 1) and a CpG oligonucleotide (Group SQ-2 and Group IN-5) or with gD antigen plus biphasic lipid vesicles (formulation no. 2) and a CpG oligonucleotide (Group SQ-3 and Group IN-6).

FIG. 4 is a bar graph showing the anti-Gap C IgG serum titre in naive mice (Group 4-1) or mice immunized with a bacterial antigen isolated from Gap C of Streptococcus uberis (herein “Gap C antigen”) plus a CpG oligonucleotide (Group 4-2); or with Gap C antigen plus a CpG oligonucleotide plus one of two different biphasic lipid vesicles formulations (Groups 4-3 and 4-4).

### DETAILED DESCRIPTION OF THE INVENTION

I. Definitions

The following terms as used herein shall have the following meanings.

“Antigen” refers to a substance or material that is recognized specifically by an antibody and/or combines with an antibody.

“Adjuvant” refers to a substance or material that potentiates an immune response when administered in conjunction with an antigen. An adjuvant can also be used to elicit an immune response more rapidly.

“Biphasic lipid vesicles” refer to lipid particles formed of a vesicle-forming lipid and having an oil-in-water emulsion in the central core compartment. The terms lipid vesicle, vesicle, and biphasic lipid vesicle are used herein interchangeably.

“Immunogen” refers to a substance or material, including an antigen, that is capable of inducing an immune response. Immunogens can elicit immune responses either alone or in combination with an adjuvant. An immunogen can be synthetic or natural and can be, for example, an inorganic or organic compound such as a hapten, a protein, peptide, polysaccharide, nucleoprotein, nucleic acid or lipoprotein. Immunogens may be derived from a bacterial, viral or protozoal, plant, or fungal organism or fractions thereof.

“Dose” refers to the amount of immunogen needed to elicit an immune response. The amount varies with the animal, the immunogen, and the presence of adjuvant, as described hereinbelow. The immunization dose is readily determined by methods known to those of skill in the art, such as through host animal immunization and challenge studies (Chanock, et al., (1987)).

A “CpG oligonucleotide” intends a oligonucleotide having a sequence including at least the following formula:

5′X₁CGX₂3′

where X₁ and X₂ are nucleotides and the oligonucleotide includes at least 4 nucleotides. In a preferred embodiment, C and/or G is unmethylated.

II. Immunostimulatory Composition

In one aspect, the invention includes a composition for enhancing the immune response of an antigen or an immunogen. The basic component of the composition is a biphasic lipid vesicle. Biphasic lipid vesicles have been described in the art, for example, in U.S. Pat. Nos. 5,853,755 and 5,993,852, which are incorporated by reference herein. The vesicle is administered in combination with an immunogen, where the immunogen can be entrapped in the vesicles or simply added to the external suspension media in which the vesicles are contained. As used herein, an immunogen is “associated” with biphasic lipid vesicles when the immunogen is entrapped in the vesicles or is admixed with the lipid vesicles in such a way that the immunogen is contained in the medium in which the vesicles are suspended.

A. Biphasic Lipid Vesicles

The biphasic lipid vesicles of the present invention include in the central core compartment of the lipid vesicle, and in the aqueous space separating the lipid bilayers, an oil-in-water emulsion. In general, such lipid vesicles are prepared by mixing an oil-in-water emulsion with vesicle-forming lipids. Importantly, the oil-in-water emulsion is stabilized with a surfactant prior to mixing with the vesicle-forming lipids. That is, the oil droplets in the emulsion are surrounded by a surfactant, preferably, surrounded by a monolayer of surfactant. In a preferred embodiment, the stabilizing surfactant is other than the vesicle-forming lipid component forming the biphasic lipid vesicle bilayers.

More specifically, biphasic lipid vesicles in accordance with the present invention are prepared according to the general procedure described in Example 1. The selected lipid components are solubilized in a suitable solvent, which in a preferred embodiment, is a pharmaceutically acceptable hydrophilic solvent, such as a polyol, e.g., propylene glycol, ethylene glycol, glycerol, or an alcohol, such as ethanol, or mixtures of such solvents. Depending on the physicochemical properties of the lipid components and on the selected solvent, it may be necessary to warm the mixture, for example, to between 40-80° C.

The lipid components necessarily include a vesicle-forming lipid, by which is meant an amphipathic lipid having a hydrophobic tail and a head group which can form spontaneously into bilayer vesicles in water. The vesicle-forming lipids are preferably ones having two hydrocarbon chains, typically acyl chains, and where the head group is either polar or nonpolar. There are a variety of synthetic vesicle-forming lipids and naturally-occurring vesicle-forming lipids suitable for use, such as phospholipids, which include phosphatidylcholine, phosphatidylethanolamine, phosphatidylserine, phosphatidylinositol, phosphatidic acid, and sphingomyelin, where the two hydrocarbon chains are typically between about 14-22 carbon atoms in length, and have varying degrees of unsaturation. These lipids can be obtained commercially or prepared according to published methods.

In addition to the vesicle-forming lipid component, the lipid vesicles of the present invention can include other lipid components capable of being stably incorporated into lipid bilayers, with their hydrophobic moieties in contact with the interior, hydrophobic region of the bilayer membrane, and their polar head groups oriented toward the exterior, polar surface of the membrane. For example, glycolipids, ceramides and sterols, such as cholesterol, coprostanol, cholestanol and cholestane, long chain fatty acids (C₁₆ to C₂₂), such as stearic acid, can be incorporated into the lipid bilayer. Other lipid components that may be used include fatty amines, fatty acylated proteins, fatty acylated peptides, oils, fatty alcohols, glyceride esters, petrolatum and waxes. It will also be appreciated that a skin permeation enhancer can be included in the lipid vesicle lipid components, as will be further discussed below.

The oil-in-water emulsion is prepared by dissolving a surfactant in water or in oil, depending on the hydrophilic-lipophilic balance (HLB) of the surfactant. In a preferred embodiment, the surfactant is mixed with distilled water and added to an oil phase for formation of an emulsion. The emulsion can be formed using agitation such as homogenization or emulsification, or can be formed by micro-emulsion techniques which do not involve agitation. The resulting emulsion preferably has water as the continuous phase and oil as the dispersed phase.

The oil-in-water emulsion is stable by virtue of the oil droplets in the dispersed phase being surrounded by the surfactant. That is, the hydrophilic portion of each surfactant molecule extends into the aqueous phase of the emulsion and the hydrophobic portion is in contact with the lipophilic droplet. Lipid vesicles are formed by blending the oil-in-water emulsion with vesicle-forming lipids. If the emulsion is not surfactant-stabilized prior to contact with the vesicle-forming lipids, the vesicle-forming lipids may act to first stabilize the emulsion rather than form lipid bilayers around the oil-in-water emulsion.

Surfactants suitable for formation of the oil-in-water emulsion are numerous, including both cationic, anionic and nonionic or amphoteric surfactants. In one embodiment, the preferred surfactant is a cationic surfactant, such as linoleamidopropyl propylene glycol-dimonium chloride phosphate, cocamidopropyl propylene glycol-dimonium chloride phosphate and stearamido propylene glycol-dimonium chloride phosphate. These are synthetic phospholipid complexes commercially available from Mona Industries, Inc (Patterson, N.J.) sold under the tradenames Phospholipid EFA™ Phospholipid SV™ and Phospholipid SVC™, respectively. Another preferred vesicle-forming lipid for use as the primary lipid component of the biphasic lipid vesicle bilayers is hydrogenated phosphatidylcholine.

Exemplary anionic surfactants include acylglutamates, such as triethanolamine-cocoyl glutamate, sodium lauroyl glutamate, sodium hydrogenated tallow glutamate and sodium cocoyl glutamate.

Exemplary nonionic surfactants include naturally derived emulsifiers, such as polyethyleneglycol-60 almond glycerides, avocado oil diethanolamine, ethoxylated jojoba oil (polyethyleneglycol-40 jojoba acid and polyethyleneglycol-40 jojoba alcohol); polyoxyethylene derivatives, such as polyoxyethylene-20 sorbitan monooleate and polyoxythethylene-20 sorbitan monostearate; lanolin derivatives, such as polychol 20 (LANETH 20) and polychol 40 (LANETH 40); and neutral phosphate esters, such as polypropyleneglycol-cetyl ether phosphate and diethanolamine oleth-3 phosphate.

The oil droplets in the dispersed oil phase preferably have sizes of less than about 1 μm, more preferably less than about 0.5 μm, in diameter. The droplet size, of course, is readily adjusted by mixing conditions, e.g., shear and time of mixing, etc.

It will be appreciated that other components can be added to the oil-in-water emulsion, that is, the oil-in-water emulsion need not be of oil, surfactant and water alone. For example, the emulsion can include antimicrobial agents, such as methylparaben, propylparaben, and enhancing ingredients such as waxes, fatty alcohols, fatty acid esters, glyceryl stearate, petrolatum, plant oils and extracts, and combinations thereof. Specific preferred examples include beeswax, olive oil, glyceryl stearate, cetyl alcohol, stearyl alcohol, myristyl myristate, and cetyl palmitate, stearyl heptanoate, and stearyl palmitate. Exemplary formulations suitable for use in the present invention are described below in Examples 1 and 3.

The stabilized oil-in-water emulsion is mixed with the solubilized vesicle-forming lipid and, if added, other lipid components, e.g., cholesterol. The emulsion and the lipid components are mixed under conditions effective to form multilamellar vesicles having in the central compartment the oil-in-water emulsion.

The size of the vesicles is typically between about 0.1-100 m. For use in the present invention, a lipid vesicle size of between about 0.5-25 μm is preferred, which can be most readily obtained by adjusting the mixing conditions.

The composition of lipid vesicles formed in accordance with the invention have a consistency similar to a cream without further addition of thickening or gelling agents. The consistency is readily adjustable according to the desired mode of administration. For example, for subcutaneous administration or intravenous administration, a thinner consistency may be desired than that used for topical administration. The consistency for intranasal and inhalation administration can also be adjusted accordingly.

The population of vesicles formed according to the technique described in Example 1 has a uniform size distribution and homogeneous composition. The vesicles are physically stable, that is, little aggregation or fusion of vesicles is evident after storage for a four year period.

B. CpG Oligonucleotide Adjuvant

As noted above, in one embodiment, the composition includes an oligonucleotide having at least one cytosine-guanine dinucleotide (CpG). As will be described below, studies performed in support of the invention show that the immune response achieved by administration of an immunogen in combination with a biphasic lipid vesicle composition can be further enhanced by including a CpG oligonucleotide in the biphasic lipid vesicle—immunogen composition.

DNA motifs consisting of an unmethylated CpG dinucleotide flanked by two 5′ purines and two 3′ pyrimidines stimulate an innate immune response characterized by the production of IgM, IFNγ, IL-6, IL-12, IL-18, and TNFα (Klinman et al., Kreig et al.). These sequence motifs are 20 times more common in microbial than mammalian DNA due to differences in the frequency of utilization and the methylation pattern of CpG dinucleotides in prokaryotes versus eukaryotes.

In a preferred embodiment, the immunostimulatory CpG nucleic acid contains a consensus mitogenic CpG motif represented by the formula:

5′X₁CGX₂3′

where X₁ and X₂ are nucleotides. In a preferred embodiment, C and/or G is unmethylated. In another embodiment X₁ is selected from A, G, and T and X₂ is C or T. More generally, the CpG oligonucleotide is of the form:

NₙX₁CGX₂Nₘ

where X₁, X₂, and N, are nucleotides, and n and m individually range from 0 to about 100. Thus, the CpG nucleic acid is preferably a nucleic acid sequence having between about 2-250 base pairs, and in a more preferred embodiment is a oligonucleotide having at least 4 base pairs. A preferred range for the CpG oligonucleotide is between about 4-100 base pairs, and more preferably between about 8-40 nucleotides.

In studies performed in support of the invention, the CpG oligonucleotide identified herein as SEQ ID NO:1 (TCCATGACGTTCCTGACGTT) was used as part of a composition comprised of biphasic lipid vesicles and an antigen. As will be shown, the CpG oligonucleotide and the vesicles act synergistically to achieve an enhanced immune response, relative to the response achieved when vesicles alone or the oligonucleotide alone are administered. It will be appreciated that a variety of CpG oligonucleotides are suitable for use. For example, oligonucleotide sequences of any length comprising one or more of the following sequences are exemplary: TCAACGTT (SEQ ID NO:2), GACGTT (SEQ ID NO:3), AGCGTT (SEQ ID NO:4), AACGCT (SEQ ID NO:5), or AACGAT (SEQ ID NO:6), wherein C and G are unmethylated. In another embodiment, a T nucleotide adjacent to any one of these sequences is contemplated, where the T is added on the 5′ end to yield, for example, TTCAACGTT (SEQ ID NO:7), TGACGTT (SEQ ID NO:8), TAGCGTT (SEQ ID NO:9), TAACGCT (SEQ ID NO:10), and TAACGAT (SEQ ID NO:11). Other suitable sequences are described in the art (see, for example, U.S. Pat. Nos. 6,214,806; 6,207,646; 6,239,116; 6,218,371).

It will also be appreciated that the CpG oligonucleotide can have a phosphate backbone modification, such as a phosphorothioate backbone modification.

C. Antigen

In general, a wide variety of immunogens are suitable for use in the present invention. The following list of antigens is provided by means of illustration and is not meant to be exclusive: influenza virus antigens (such as haemagglutinin and neuraminidase antigens), Bordetella pertussis antigens (such as pertussis toxin, filamentous haemagglutinin, pertactin), human papilloma virus (HPV) antigens, Helicobacter pylon antigens, rabies antigens, tick-borne encephalitis (TBE) antigens, meningoccal antigens (such as capsular polysaccharides of serogroup A, B, C, Y and W-135), tetanus antigens (such as tetanus toxoid), diphtheria antigens (such as diphtheria toxoid), pneumococcal antigens (such as Streptococcus pneumoniae type 3 capsular polysaccharide), tuberculosis antigens, human immunodeficiency virus (HIV) antigens (such as GP-120, GP-160), cholera antigens (such as cholera toxin B subunit), staphylococcal antigen (such as staphylococcal enterotoxin B), shigella antigens (such as shigella polysaccharides), vesicular stomatitis virus antigen (such as vesicular stomatitis virus glycoprotein), cytomegalovirus (CMV) antigens, hepatitis antigens (such as hepatitis A (HAV), B (HBV), C (HCV), D (HDV) and G (HGV) virus antigens, respiratory syncytial virus (RSV) antigens, herpes simplex antigens, or combinations thereof (e.g., combinations of diphtheria, pertussis and tetanus (DPT)). Suitable antigens also include those delivered for induction of tolerance, such as retinal antigens. Antigens for immunization/vaccination against anthrax and Yersinia pestis are also contemplated.

Preferred antigens include Bordetella pertussis antigens, meningococcal antigens, tetanus antigens, diphtheria antigens, pneumococcal antigens, tuberculosis antigens, and RSV antigens. In another preferred embodiment, the entrapped immunogen has a molecular weight of between about 100-100,000,000 daltons, more preferably 100-500,000 daltons, and most preferably 100-100,000 daltons.

In studies performed in support of the present invention, an antigen isolated from the outer membrane of Actinobacillus pleuropneumoniae (OmIA) was used as a model antigen, as will be described below.

III. Administration of Exemplary Compositions

In studies performed in support of the invention, the ability of biphasic lipid vesicles to enhance the immune response to an antigen referred to herein as “OmIA” was evaluated. OmIA is an antigen isolated from the outer membrane of lipoprotein A in Actinobacillus pleuropneumoniae. The ability of the composition to confer protection to a challenge with A. pleuropneumoniae was also evaluated. The study also evaluated the effect of administering a CpG oligionucleotide in combination with the biphasic lipid vesicles.

As described in Example 2, biphasic lipid vesicles composed of “Formulation No. 1” (see Example 1) were prepared. The vesicles admixed with OmIA were administered subcutaneously to pigs two times at a three-week interval. Some pigs also received, admixed with the vesicles and the OmIA, a CpG oligonucleotide (SEQ ID NO:1). As controls, a group of pigs received the OmIA antigen in combination with lipid vesicles and an oligonucleotide sequence similar to SEQ ID NO:1 but containing no CpG motifs. This control sequence is referred to herein as SEQ ID NO:12 and has the sequence TCCAGGACTTCTCTCAGGTT. The test groups and formulations are summarized in Table 1.

TABLE 1
Formulations used in Experiment 1.

| Group 1-1 | | | Group 1-4 |
|---|---|---|---|
| (control) | Group 1-2 | Group 1-3 | (control) |
| 0.5 ml saline | 0.5 ml biphasic lipid | 0.5 ml biphasic lipid | 0.5 ml biphasic lipid |
| | vesicles | vesicles | vesicles |
| | OmlA¹ 50 μg | OmlA 50 μg | OmlA 50 μg |
| | | CpG oligo (SEQ ID | Non-CpG oligo |
| | | No:1) 1 mg | (SEQ ID NO:12) 1 |
| | | | mg |

¹OmlA = antigen isolated from the outer membrane of lipoprotein A in Actinobacillus pleuropneumoniae.

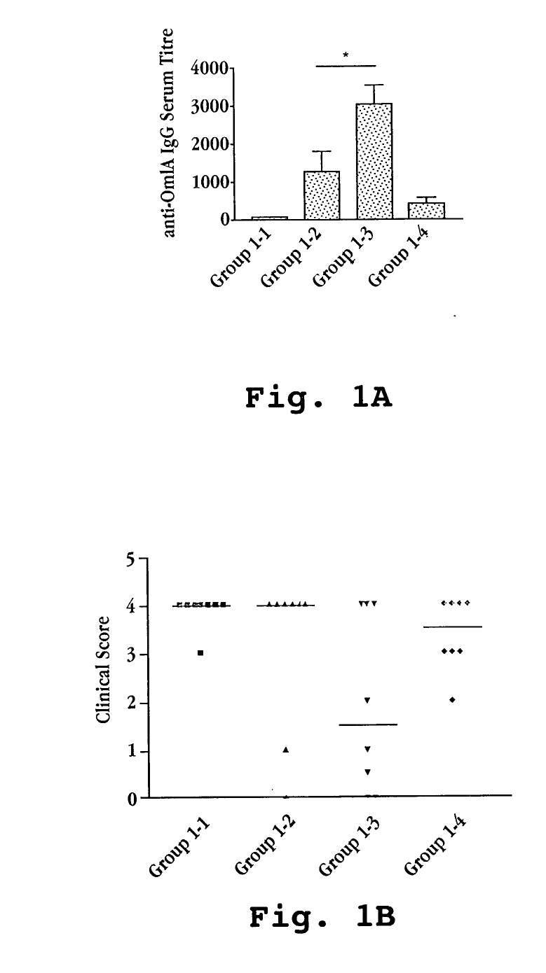

Ten days after the second immunization, OmIA-specific IgG was determined in the serum and the pigs were challenged with A. pleuropneumoniae by inhalation. Five days after the challenge, clinical scores were taken, a quantification of bacterial isolation was done, and a postmortem examination was performed. The results are shown in FIGS. 1A-1B and Table 2.

FIG. 1A is a bar graph showing the anti-OmIA IgG serum titre in the animals in each test group. Pigs immunized with OmIA admixed with biphasic lipid vesicles (Group 1-2) had an enhanced immune response when compared to pigs treated with saline alone (Group 1-1). Addition of a CpG oligonucleotide to the vesicle-antigen composition achieved a further stimulation of immune response, as evidenced by comparing the results for Group 1-3 and Group 1-2. That is, animals treated with biphasic lipid vesicles plus a CpG oligonucleotide (Group 1-3) had a significantly higher OmIA-specific IgG titer than did the animals treated with biphasic lipid vesicles alone (Group 1-2) or than animals immunized with the control composition of a non-CpG oligonucleotide and vesicles (Group 1-4).

As noted above, ten days after the second immunization, all animals were exposed to a challenge of A. pleuropneumoniae by inhalation in a chamber. The severity of lung lesions was recorded at autopsy five days post-challenge or, in pigs with severe infection, the examination and bacterial isolation were done at the time of euthanasia. The results are shown in FIG. 1B.

FIG. 1B shows the lung pathology score for each animal in each test group, where the proportion of lung with pneumonic lesions was determined as the portion of dorsal and ventral surfaces of the lungs with gross lesions. Group 1-1 is represented by the closed squares, Group 1-2 by the closed triangles, Group 1-3 by the inverted closed triangles, and Group 1-4 by the closed diamonds. Each point in the Figure represents one animal. As seen, the animals in Group 1-3 immunized with OmIA in the presence of biphasic lipid vesicles and the CpG oligonucleotide showed fewer lung lesions (as evidenced by the lowest average lung pathology clinical score). The solid line in the Group 1-3 data points represents the average score for the test animals in the group.

Bacteria counts were also determined in the lungs and lymph nodes of the animals in each test group. Table 2 summarizes the A. pleuropneumoniae isolated from lung lesions or lymph nodes in the challenged pigs, where a “−” symbol represents no observed bacterial isolates and the “+”, “++”, and “+++” symbols correspond to a progressively greater number of observed isolates.

TABLE 2

| Group 1-1 | | | Group 1-2 | | | Group 1-3 | | | Group 1-4 | | |
|---|---|---|---|---|---|---|---|---|---|---|---|
| Pig #. | LN¹ | Lung | Pig # | LN¹ | Lung | Pig # | LN¹ | Lung | Pig # | LN¹ | Lung |
| 21 | ++ | +++ | 29 | + | + | 37 | ++ | − | 45 | ++ | − |
| 22 | + | + | 30 | + | − | 38 | ++ | + | 46 | + | − |
| 23 | ++ | +++ | 31 | +++ | + | 39 | + | − | 47 | ++ | − |
| 24 | + | − | 32 | ++ | − | 40 | + | − | 48 | + | − |
| 25 | + | − | 33 | + | − | 41 | + | − | 49 | ++ | ++ |
| 26 | ++ | +++ | 34 | + | +++ | 42 | + | ++ | 50 | ++ | +++ |
| 27 | | | 35 | ++ | +++ | 43 | + | − | 51 | + | +++ |
| 28 | ++ | +++ | 36 | | | 44 | ++ | ++ | 52 | ++ | ++ |

¹LN = lymph node

As seen in Table 2, pigs immunized with OmIA in the presence of biphasic lipid vesicles and a CpG oligonuclotide (Group 1-3) showed fewer bacteria isolated from the lungs and lymph nodes when compared to pigs immunized with lipid vesicles alone (Group 1-2) or lipid vesicles plus the control non-CpG oligonucleotide (Group 1-4).

This study demonstrated that immunization of animals with a biphasic lipid vesicle formulation alone achieved an immune response. That immune response was further enhanced by additionally administering a CpG oligonucleotide. The study also showed that more animals in the group treated with both lipid vesicles and a CpG oligonucleotide were protected against infection to a greater extent than animals immunized with a biphasic lipid vesicle formulation in the absence of a CpG oligonucleotide or with a biphasic lipid vesicle formulation and a non-CpG oligonucleotide.

In another study performed in support of the invention, the ability of the biphasic lipid vesicle formulation to enhance the adjuvant activity of CpG oligonucleotides was evaluated. In addition, the enhancement of immune response achieved by the biphasic lipid vesicle formulation was compared to that offered by a mineral-oil based adjuvant.

In this study, the experimental procedures described for the study above (and set forth in Example 2) were followed. The four groups of test animals (pigs, n=8) were immunized as follows:

Group 2-1: Control, 0.5 ml phosphate buffered saline;

Group 2-2: 0.5 ml phosphate buffered saline; 50 μg OmIA; and 1 mg CpG oligonucleotide SEQ ID NO:1;

Group 2-3: 0.5 ml biphasic lipid vesicles (formulation no. 1); 50 μg OmIA; and 1 mg CpG oligonucleotide SEQ ID NO:1; and

Group 2-4: 0.5 ml mineral oil-based commercial adjuvant and 50 μg OmIA.

All animals were immunized subcutaneously two times at a three week interval. As in the previous study, ten days after the second immunization, the OmIA-specific IgG was determined in the serum by the ELISA assay set forth in Example 2. The results are shown in FIG. 2.

FIG. 2 is a bar graph showing the anti-OmIA IgG in serum for each of the test groups. As seen by comparing the response of Group 2-2 with that of Groups 2-3, the biphasic lipid vesicle formulation enhanced the adjuvant activity of CpG oligonucleotide. The responses induced by the biphasic lipid vesicle plus CpG oligonucleotide formulation were comparable to the response induced by commercial mineral-based adjuvant. However, histological assessment of the site of injection showed that administration of the biphasic lipid vesicles and CpG oligonucleotide induced no or mild inflammation, in contrast to the severe inflammation caused by the commercial adjuvant (as evidenced by infiltration of mononuclear cells and necrosis at the immunization site, data now shown).

Example 3 describes another study performed in support of the present invention where a mouse model was used to show the enhanced immune response achieved when biphasic lipid vesicles are administered in combination with a CpG oligonucleotide. In this study, two different biphasic lipid vesicle formulations were evaluated and the composition of each is described in Table 3 of Example 3.

Mice were randomized into six test groups and immunized subcutaneously (SQ) or intranasally (IN) with the viral antigen glycoprotein D (“gD antigen”) of herpes simplex virus type 1 (HSV-1) as follows:

Group SQ 3-1: Control, “gD” antigen alone in saline;

Group SQ 3-2: biphasic lipid vesicles (formulation no. 1), CpG oligonucleotide (SEQ ID NO:1); and antigen “gD”;

Group SQ 3-3: biphasic lipid vesicles (formulation no. 2), CpG oligonucleotide (SEQ ID NO:1); and antigen “gD”;

Group IN 3-4: Control, “gD” antigen alone in saline;

Group IN 3-5: biphasic lipid vesicles (formulation no. 1), CpG oligonucleotide (SEQ ID NO:1); and antigen “gD”;

Group IN 3-6: biphasic lipid vesicles (formulation no. 2), CpG oligonucleotide (SEQ ID NO:1); and antigen “gD”;

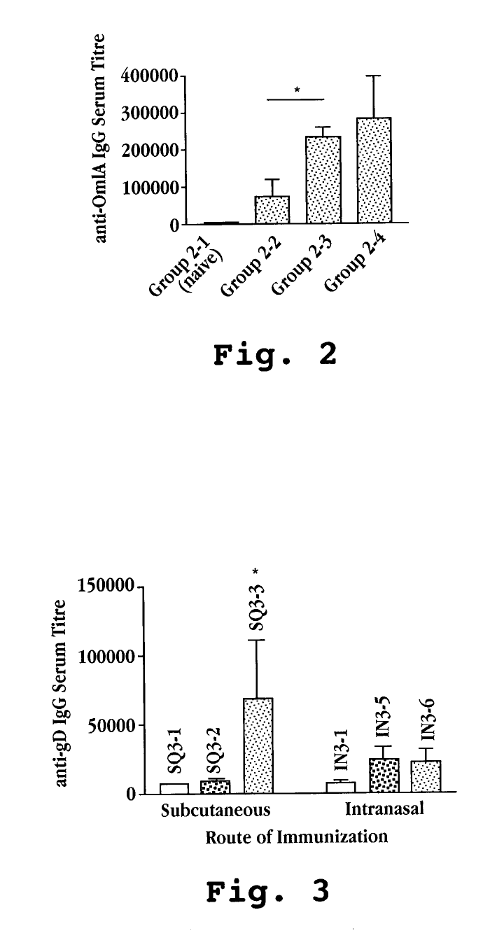

Animals were reimmunized by the same route as initially immunized two weeks later. Ten days after this booster, serum was collected. The anti-gD IgG in the serum was determined and the results are shown in FIG. 3.

FIG. 3 is a bar graph showing the anti-gD IgG serum titre for the six test groups. The mice immunized with the biphasic lipid vesicle formulations in combination with the CpG oligonucleotide had an enhanced immune response. In particular, mice immunized with the lipid vesicle formulation no. 2 (Groups SQ 3-3 and IN 3-6) had a significantly enhanced immune response compared to the mice treated with the antigen alone (Groups SQ 3-1 and IN 3-4).

In yet another study described in Example 4, mice were immunized subcutaneously with the bacterial antigen Gap C of Streptococcus uberis (herein “Gap C antigen”). Mice were randomized into four treatment groups for immunization as follows:

Group 4-1: Control, naïve mice;

Group 4-2: Gap C antigen plus CpG oligonucleotide (SEQ ID NO:1);

Group 4-3: Gap C antigen plus CpG oligonucleotide (SEQ ID NO:1) plus biphasic lipid vesicles (formulation no. 1);

Group 4-4: Gap C antigen plus CpG oligonucleotide (SEQ ID NO:1) plus biphasic lipid vesicles (formulation no. 2).

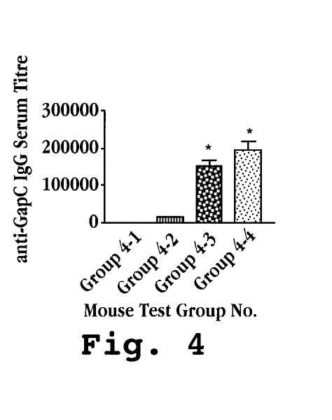

Animals were reimmunized two weeks later. Ten days after this booster, serum was collected. The anti-Gap C IgG in the serum was determined and the results are shown in FIG. 4.

FIG. 4 is a bar graph showing the anti-Gap C IgG serum titre in each of the test groups. As seen, the mice immunized with Gap C in the presence of both biphasic lipid vesicles and a CpG oligonucleotide (Group 4-3 and Group 4-4) had an enhanced immune response when compared to animals immunized with Gap C and a CpG oligonucleotide alone (Group 4-2).

These studies show that a CpG oligonucleotide associated with a biphasic lipid vesicle composition gives a synergistically enhanced immune response. Enhanced immune response were observed when immunization was by the intranasal route or the subcutaneous route.

IV. Methods and Products for Administration

In another aspect, the invention includes a method of enhancing the immune response elicited by an immunogen by administering a biphasic lipid vesicle composition in combination with a CpG oligonucleotide. The lipid vesicle and oligonucleotide and antigen components can be admixed together to form a mixture of the three, or one or both of the antigen and the oligonucleotide can be entrapped in the lipid vesicles. Entrapping either the oligonucleotide or the antigen in the vesicles is readily done by those of skill in the art, typically by mixing the component with either the lipid phase or with the oil or water phase of the emulsion prior to vesicle formation.

It will be appreciated that the method contemplates administration by any suitable route, including but not limited to subcutaneous, intravenous, intramuscular, topical, intranasal, inhalation, mucosal (buccal, vaginal) and the like.

In another aspect, the invention includes a kit for preparing a composition for immunization of a subject. In one embodiment, the kit includes (i) a biphasic lipid vesicle component; (ii) an immunogen component; and (iii) an oligonucleotide component, the oligonucleotide having at least one cytosine-guanine (CpG) dinucleotide, e.g, a CpG oligonucleotide. The three components are admixed to form a composition suitable for administration to a subject by any desirable route. The composition is capable of eliciting an immune response to the immunogen.

In another embodiment, the kit is comprised of (i) a biphasic lipid vesicle-entrapped immunogen component; and (ii) a CpG oligonucleotide component. The two components are admixed to form a composition effective to elicit an immune response.

In another embodiment, the kit is comprised of (i) a biphasic lipid vesicle-entrapped CpG oligonucleotide; and (ii) an immunogen component. The two components are admixed to form a composition that upon administration is effective to elicit an immune response.

V. Examples

The following examples further illustrate the invention described herein and are in no way intended to limit the scope of the invention.

### EXAMPLE 1

### Preparation of Biphasic Lipid Vesicles

A. Preparation of Lipid Components

Lipid components, hydrogenated phosphatidylcholine (Phospholipon 90H™, Natterman GmbH, Germany) and cholesterol, were mixed in the amounts shown in Table 2 with propylene glycol and mixed with warming to between about 65-75° C.

B. Preparation of Oil-In-Water Emulsion

An oil-in-water emulsion was prepared by mixing the surfactant TWEEN 8™ with methylparaben and propylparaben, in the amounts shown in Table 4, in distilled water.

In a separate container, the lipophilic components, canola oil and Poloxamer 407™, were blended together.

The water phase and the oil phase were mixed together in a high pressure homogenizer (H-5000 Laboratories Homogenizer Microfluidic Corp.). Visually, the emulsion is a milky solution having the consistency of water.

C. Biphasic Lipid Vesicle Formation

The lipid components and the oil-in-water emulsion were mixed together by vortexing or propeller mixing at 50-300 rpm. This formulation is referred to herein as “Formulation No. 1”.

TABLE 4
Composition of “Formulation No. 1”

| Component | % (w/w) |
|---|---|
| Hydrogenated phosphatidylcholine | 2 |
| Cholesterol | 0.2 |
| Propylene glycol | 2 |
| Tween 80 ™ | 0.1 |
| Methylparaben | 0.15 |
| Propylparaben | 0.05 |
| Canola oil | 1 |
| Poloxamer 407 ™ | 1 |
| Distilled water | q.s. to 100 |

### EXAMPLE 2

### In vivo Administration of Lipid Vesicle-CpG Composition

Lipid vesicles were prepared as described in Example 1.

An antigen isolated from the outer membrane of lipoprotein A of Actinobacillus pleuropneumoniae (designated herein “OmIA”) was selected as a model antigen.

CpG oligonucleotide identified herein as SEQ ID NO:1 was used as a model CpG oligonucleotide. A sequence of the same length and identical but for two nucleotide substitutions to destroy the CpG motif was used as a control sequence to control for any effect due to the nucleic acid, and this sequence is identified herein as SEQ ID NO:12. Both oligionucleotides had a phosphorothioate backbone modification to increase resistance to nuclease degradation.

Four-week old male and female pigs were obtained from a herd free of Actinobacillus pleuropneumoniae. Thirty-two pigs were randomized into four test groups (n=8). All the animals received two immunizations 21 days apart as follows:

Group 1-1: Control, 0.5 ml phosphate buffered saline;

Group 1-2: 0.5 ml biphasic lipid vesicles (formulation no. 1) and 50 μg OmIA;

Group 1-3: 0.5 ml biphasic lipid vesicles (formulation no. 1); 50 μg OmIA; and 1 mg CpG oligonucleotide (SEQ ID NO:1); and

Group 1-4: 0.5 ml biphasic lipid vesicles (formulation no. 1); 50 μg OmIA; and 1 mg non-CpG oligonucleotide (SEQ ID NO:12).

These test groups and the administered composition are summarized in Table 1 above.

Ten days after the last immunization serum samples were taken to evaluate the induction of antigen-specific humor immune response, by analyzing for OmIA-specific IgG levels in the serum. OmIA-specific serum antibodies were determined by ELISA as previously described (Gerlach G. F. et al, Infect. Immun., 61:565-72 (1993)). Briefly, ninety-six well plates (Immulon 2; Dynatech Laboratories Inc., Alexandria, Va.) were coated with OmIA (1 μg/ml) in a carbonate-bicarbonate buffer (pH 9.6). Plates were incubated overnight at 4° C. and then washed 4 times in PBS containing 0.05% Tween™ (PBS-T). Four-fold dilutions of sera were prepared in PBS-T (containing 0.5% gelatin) and dispensed in 100 μl volumes. Alkaline phosphatase goat anti-porcine IgG(H+L) conjugate (KPL, Gaithersburg, Md.) was used as the detecting antibody. After incubation for one hour and four subsequent washes, Di(Tris) p-nitrophenyl phosphate (Sigma, Oakville, ON) was used as the chromogenic substrate. The absorbance was read after 15-20 minutes at 405 nm (BIO-RAD, Richmond, Calif.). Titres are expressed as the reciprocal of the highest dilution with an O.D of three standard deviations above the negative control and are shown in FIG. 1A.

A. Actinobacillus pleuropneumoniae Challenge

Ten days after the last immunization and after the serum samples were drawn (see above) the pigs were challenged by exposure to an aerosol generated from a suspension of 1.5×10⁵ CFU/mL of App serotype 1 (Willson P. J. et al., Cancer J. Vet. Res., 65:206-12 (2001); Gerlach G. F. et al, Infect. Immun., 61:565-72 (1993)). Briefly, an aerosol of bacteria was generated with a Devilbis 65 nebulizer into a Plexiglass and steel chamber where pigs were allowed to breathe the mist for ten minutes (Osborne, A. D., et al., Cancer J. Comp. Med., 49:434 (1985)). A veterinarian and an animal health technician daily evaluated clinical signs of disease in all pigs. The following ordinal scoring system was used: clinically normal (0); slight increase in respiratory rate and effort with slight depression (1); marked increase in respiratory rate and effort with marked depression (2); severe increase in respiratory rate and effort with severe depression, mouth breathing and/or cyanosis (3). Pigs with a clinical score of 3 were humanely killed. On day five after challenge all remaining pigs were humanely killed, and examined postmortem. The proportion of lung with pneumonic lesions was determined as the portion of dorsal and ventral surfaces of the lungs with gross lesions of pneumonia. The results are shown in FIG. 1B.

### EXAMPLE 3

### Immunization with Glycoprotein D of Herpes Simplex Virus Type 1

A. Lipid Vesicle Preparation

Lipid vesicles were prepared as described in Example 1 with the following changes to the formulation, to result in a formulation referred to herein as “Formulation No. 2”.

The oil-in-water emulsion was prepared by mixing the surfactant linoleamidopropyl propylene glycol-dimonium chloride phosphate (Phospholipid EFA™, Mona Industries Inc., Patterson, N.J.), methylparaben and propylparaben, in the amounts shown in Table 1, in distilled water.

In a separate container, the lipophilic components Mygliol 810N (caprylic/capric triglyceride) and glycerol monostearate were blended together.

The components and amounts of Formulation No. 2 are shown in Table 5.

TABLE 5
Composition of “Formulation No. 2”

| Component | % (w/w) |
|---|---|
| Hydrogenated phosphatidylcholine | 2 |
| Cholesterol | 0.2 |
| Propylene glycol | 2 |
| Phospholipid EFA ™ | 2 |
| Methylparaben | 0.15 |
| Propylparaben | 0.05 |
| Mygliol 810N ™ | 1 |
| Glycerol monostearate | 1 |
| Distilled water | qs to 100 |

B. Oligonucleotides

The CpG oligonucleotide identified herein as SEQ ID NO:1 and the non-CpG oligonucleotides identified herein as SEQ ID NO:12 were used at 10 μg per subcutaneous immunization and 1 μg per mucosal immunization. Both these oligonucleotides contain a nuclease resistant phosphorothioate backbone.

C. Antigen and Antigen Delivery System

Viral antigen glycoprotein D “gD” of herpes simplex virus type 1 (HSV-1) in endotoxin-free saline was mixed with the biphasic lipid formulation no. 2 at a ratio of 1 part antigen to 9 parts lipid vesicle formulation.

D. In vivo Immunization

Six week-old female BALB/c mice were used for the study, with five mice in each group. The mice were immunized by the intranasal or subcutaneous route with 0.5 μg of viral antigen glycoprotein D “gD” of herpes simplex virus type 1 (HSV-1) in a volume of 100 μL. The formulation test groups were as follows:

Group SQ 3-1: Control, “gD” antigen in saline

Group SQ 3-2: biphasic lipid vesicles (formulation no. 1), CpG oligonucleotide (SEQ ID NO:1, 10 μg); and antigen “gD” (0.5 μg)

Group SQ 3-3: biphasic lipid vesicles (formulation no. 2), CpG oligonucleotide (SEQ ID NO:1, 10 μg); and antigen “gD” (0.5 μg)

Group IN 3-4: Control, “gD” antigen (0.5 mg) alone in saline

Group IN 3-5: biphasic lipid vesicles (formulation no. 1), CpG oligonucleotide(SEQ ID NO:1, 1 μg); and antigen “gD” (0.5 μg)

Group IN 3-6: biphasic lipid vesicles (formulation no. 2), CpG oligonucleotide(SEQ ID NO:1, 1 μg); and antigen “gD” (0.5 μg)

Animals were re-immunized two weeks later and serum was collected 10 days after the boost. Results are shown in FIG. 3 and represent the mean titre of five mice per group, where the bar indicates the SEM.

### EXAMPLE 4

### Immunization with Bacterial Antigen Gap C of Streptococcus uberis

Mice were immunized subcutaneously with the bacterial antigen Gap C of Streptococcus uberis (herein “Gap C antigen”), similar to the procedures described above in Example 3. Mice were randomized into four treatment groups for immunization as follows:

Group 4-1: Control, naïve mice;

Group 4-2: Gap C antigen (10 μg) plus CpG oligonucleotide (SEQ ID NO:1, 10 μg);

Group 4-3: Gap C antigen (10 μg) plus CpG oligonucleotide (SEQ ID NO:1, 10 μg) plus biphasic lipid vesicles (formulation no. 1);

Group 4-4: Gap C antigen (10 μg) plus CpG oligonucleotide (SEQ ID NO:1, 10 μg) plus biphasic lipid vesicles (formulation no. 2).

Animals were re-immunized two weeks later and serum was collected 10 days after the boost. Results are shown in FIG. 4 as the mean titre of five mice per group, where the bar indicates the SEM.

Although the invention has been described with respect to particular embodiments, it will be apparent to those skilled in the art that various changes and modifications can be made without departing from the invention.

It is claimed:

1. A composition for eliciting in a subject an immune response to an immunogen, comprising
a suspension of biphasic lipid vesicles having a central core compartment containing an oil-in-water emulsion, and
associated with the vesicles, an immunogen.

2. The composition of claim 1, wherein said immunogen is admixed with the vesicles.

3. The composition of claim 1, wherein said immunogen is entrapped in the vesicles.

4. The composition of claim 1 further comprising a nucleic acid sequence having at least one cytosine-guanine (CpG) dinucleotide.

5. The composition of claim 4, wherein cytosine and guanine in the cytosine-guanine (CpG) dinucleotide are unmethylated.

6. The composition of claim 4, wherein said sequence contains a sequence selected from the sequences identified herein as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11.

7. The composition of claim 4, wherein the nucleic acid sequence comprises between about 4 to 100 nucleotides.

8. The composition according to claim 4, wherein the sequence has a phosphate backbone modification.

9. The composition of claim 8, wherein the phosphate backbone modification is a phosphorothioate backbone modification.

10. A composition for eliciting in a subject an immune response to an immunogen, comprising
a suspension of biphasic lipid vesicles having a central core compartment containing an oil-in-water emulsion, and
associated with the vesicles, (i) an immunogen and (ii) an oligonucleotide having at least one cytosine-guanine (CpG) dinucleotide.

11. The composition of claim 10, wherein said immunogen and said CpG oligonucleotide are admixed with said vesicles.

12. The composition of claim 10, wherein said immunogen is entrapped in the vesicles.

13. The composition of claim 10, wherein said CpG oligonucleotide is entrapped in the vesicles.

14. The composition of claim 10, wherein said immunogen and said CpG oligonucleotide are entrapped in the vesicles.

15. The composition of claim 10, wherein the immunogen is selected from the group consisting of antigens derived from bacterial, viral, parasitic, plant and fungal origin.

16. The composition of claim 10, wherein the oligonucleotide sequence comprises 4 to 100 nucleotides.

17. The composition of claim 10, wherein said CpG oligonucleotide comprises a sequence selected from the group consisting of sequences identified herein as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11.

18. The composition according to claim 10, wherein the oligonucleotide has a phosphate backbone modification.

19. The composition of claim 10 wherein the phosphate backbone modification is a phosphorothioate backbone modification.

20. A kit for preparation of a composition effective to elicit in a subject an immune response to an immunogen, comprising
(i) a biphasic lipid vesicle component, said vesicles having a central core compartment containing an oil-in-water emulsion,
(ii) an immunogen component; and
(iii) an oligonucleotide component, said oligonucleotide having at least one cytosine-guanine (CpG) dinucleotide;
wherein said components are admixed to form a composition effective to elicit an immune response.

21. The kit according to claim 20, wherein said CpG oligonucleotide sequence comprises between about 4 to 100 nucleotides.

22. The kit according to claim 20, wherein said CpG oligonucleotide comprises a sequence selected from the group consisting of sequences identified herein as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11.

23. The kit according to claim 20, wherein the oligonucleotide has a phosphate backbone modification.

24. The kit according to claim 20, wherein the phosphate backbone modification is a phosphorothioate backbone modification.

25. The kit according to claim 20, wherein the immunogen is selected from the group consisting of antigens derived from bacterial, viral, parasitic, plant and fungal origin.

26. A kit for preparation of a composition effective to elicit in a subject an immune response to an immunogen, comprising
(i) a biphasic lipid vesicle component, said vesicles having a central core compartment containing an oil-in-water emulsion, said vesicles containing an entrapped immunogen; and
(ii) a CpG oligonucleotide component, said oligonucleotide having at least one cytosine-guanine (CpG) dinucleotide;
wherein said components are admixed to form a composition effective to elicit an immune response.

27. The kit according to claim 26, wherein said CpG oligonucleotide sequence comprises between about 4 to 100 nucleotides.

28. The kit according to claim 26, wherein said CpG oligonucleotide comprises a sequence selected from the group consisting of sequences identified herein as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11.

29. The kit according to claim 26, wherein the oligonucleotide has a phosphate backbone modification.

30. The kit according to claim 26, wherein the phosphate backbone modification is a phosphorothioate backbone modification.

31. The kit according to claim 26, wherein the immunogen is selected from the group consisting of antigens derived from bacterial, viral, parasitic, plant and fungal origin.

32. A kit for preparation of a composition effective to elicit in a subject an immune response to an immunogen, comprising
(i) a biphasic lipid vesicle component, said vesicles having a central core compartment containing an oil-in-water emulsion, said vesicles containing an entrapped oligonucleotide having at least one cytosine-guanine (CpG) dinucleotide; and
(ii) an immunogen component;
wherein said components are admixed to form a composition effective to elicit an immune response.

33. The kit according to claim 32, wherein said CpG oligonucleotide sequence comprises between about 4 to 100 nucleotides.

34. The kit according to claim 32, wherein said CpG oligonucleotide comprises a sequence selected from the group consisting of sequences identified herein as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11.

35. The kit according to claim 32, wherein the oligonucleotide has a phosphate backbone modification.

36. The kit according to claim 32, wherein the phosphate backbone modification is a phosphorothioate backbone modification.

37. The kit according to claim 32, wherein the immunogen is selected from the group consisting of antigens derived from bacterial, viral, parasitic, plant and fungal origin.

38. An improvement in a composition comprised of a biphasic lipid vesicle and an immunogen, comprising
an oligonucleotide having at least one cytosine-guanine (CpG) dinucleotide, wherein said improvement is effective to enhance the immune response to the immunogen relative to the response obtained by administration of the vesicles and the immunogen in the absence of the oligonucleotide.

39. A method for enhancing the immune response obtained by administration of a biphasic lipid vesicle entrapped immunogen, comprising
administering an oligonucleotide having at least one cytosine-guanine (CpG) dinucleotide.
